(19) United States
(12) Patent Application Publication
(10) Pub. No.: US 2007/0111325 A1
(43) Pub. Date: May 17, 2007

(54) Methods and devices for compound screening

(76) Inventors: Marinus Gerardus Johannes Van Beuningen, Oss (NL); Herman Jacobus Blok, Retie (BE)

Correspondence Address: AMSTER, ROTHSTEIN & EBENSTEIN LLP, 90 PARK AVENUE, NEW YORK, NY, 10016, US

(21) Appl. No.: 10/580,390

(22) PCT Filed: Nov. 25, 2004
(86) PCT No.: PCT/EP04/13379

§ 371 Date: May 22, 2006

(30) Foreign Application Priority Data
Nov. 28, 2003 (EP) 03447275.3

(51) Int. Cl. G01N 33/543

(52) U.S. Cl. 436/518

(57) ABSTRACT

The present invention relates methods for screening of compounds for drug candidates comprising: (a) providing a solid porous support having first and second surfaces and at least one area with a plurality of through-going channels wherein said solid porous support comprises compounds within predefined regions of the said support; wherein said compounds within the porous structure are stored in dried condition, wherein said dried condition is obtained after a drying treatment by slow evaporation, vacuum drying or by blowing air or inert gas above and below said solid support; (b) providing a liquid sample comprising at least one molecular target; (c) mixing said dried compounds of step (a) with said liquid sample of step (b) by flow of the sample through said predefined regions of the solid support through the said through-going channels; (d) screening said compounds for drug candidates; said screening is by monitoring in an assay a compound-target interaction by measurement of a signal, said signal indicating interaction between a compound and a molecular target; (e) optionally screening for a compound having a putative effect on a drug candidate identified in step (d). The present invention further relates to the uses of said methods and solid supports and methods for the manufacture of said solid supports for carrying out said methods as well as to the use of a porous support for the preparation of a kit for carrying out said methods.

### FIELD OF THE INVENTION

The present invention relates to the field of microarray technology. In particular, the present invention relates to porous solid supports comprising compound libraries at predefined regions within the porous structure of the support.

### BACKGROUND

In a range of technology-based business sectors, including the chemical, bioscience, biomedical, and pharmaceutical industries, it is desirable to develop capabilities for rapidly and reliably carrying out chemical and biochemical reactions in large numbers using small quantities of samples and reagents.

There has been a growing interest in the development and manufacturing of microscale fluid systems for the acquisition of chemical and biochemical information. As a result of this effort, microfluidics is considered an enabling technology for providing low cost, high versality devices for drug discovery.

Very large corporate chemical and natural compound libraries have been acquired for the purpose of high throughput screening activities aimed at accelerating lead generation in drug discovery processes. These libraries are rapidly depleted by the yet extensive use of non-microscale systems that often use bulk amounts of compounds. Also with the advent of combinatorial chemistry approaches to identify pharmacologically useful compounds, it is evident that there is a need for methods and tools at microarray levels, capable of performing high-throughput characterization of pharmacological profiles and identifying corresponding potencies of the compounds in synthesized combinatorial libraries.

High-throughput 3D microarray technology has greatly reduced the volumetric quantities of test compounds and has improved the efficiency of chemical and biochemical analysis, synthesis and screening procedures.

It is therefore an object of the present invention to provide novel devices and methods for high-throughput microarray screening procedures of compounds in the discovery process of drug candidates.

### SUMMARY OF THE INVENTION

The present invention provides methods and devices for high throughput screening of compound libraries in pharmacologic assays for highly efficient lead validation and drug development.

The present invention thus provides a method for screening of compounds for drug candidates comprising:
(a) providing a solid porous support having first and second surfaces and at least one area with a plurality of through-going channels; wherein said solid porous support comprises compounds within predefined regions of the said support; wherein said compounds within the porous structure are stored in dried condition, wherein said dried condition is obtained after a drying treatment by slow evaporation, vacuum drying or by blowing air or inert gas above and below said solid support;
(b) providing a liquid sample comprising at least one molecular target;
(c) mixing said dried compounds of step (a) with said liquid sample of step (b) by flow of the sample through said predefined regions of the solid support through the said through-going channels;
(d) screening said compounds for drug candidates; said screening is by monitoring in an assay a compound-target interaction by measurement of a signal, said signal indicating interaction between a compound and a molecular target;
(e) optionally screening for a compound having a putative effect on a drug candidate identified in step (d).

The present invention also contemplates the screening of compounds for putative effects on drug candidates which may be identified according to the methods of the present invention, thereby revealing multiple combinations of drug-drug interactions.

In addition to its ability to perform highly efficient compound screening, the present invention in particular allows for storage of only minimal amounts of compounds that can be placed within the porous support structure until assay initiation.

The present invention further allows for improved conditionally-controlled and maintenance-free storage of compounds prior to the screening of said compounds in a drug discovery program.

The present invention further discloses uses of the above method according to the invention.

Additional features and advantages of the invention will be set forth in the detailed description which follows, and in part will be apparent from the description, or may be learned by practice of the invention. The objectives and other advantages of the invention will be realized and attained by the process particularly pointed out in the written description and appended claims.

### DETAILED DESCRIPTION OF THE INVENTION

Before the present methods and devices used in the method are described, it is to be understood that this invention is not limited to particular methods, components, or devices described, as such methods, components, and devices may, of course, vary. It is also to be understood that the terminology used herein is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and materials similar or equivalent to those described herein may be used in the practice or testing of the present invention, the preferred methods and materials are now described.

In this specification and the appended claims, the singular forms “a”, “an”, and “the” include plural references unless the context clearly dictates otherwise.

The present invention provides a system for high-throughput screening that is automation-friendly and allows parallel processing of numerous tests. Devices according to the present invention comprise a plate or support with an array of wells arranged in rows and columns, wherein the bottom of each well is a solid porous support having first and second surfaces and at least one area with a plurality of through-going channels. Each porous solid support in a well may comprise a microarray. The present invention therefore relates in particular to an array of arrays. It is understood by the term “test area” or “well” that these represent areas of the array which direct test compounds or other reactants or cellular components or samples onto the solid support(s). Said areas may have a depth or a height or may be planar with respect to said plate or carrier in which the individual arrays are held. Said test areas may further have any suitable shape including without limitation circular shape, square shape, rectangular shape and the like.

Each well may comprise a pool or an array of various different compounds but usually each well comprises only one compound. Accordingly, a porous solid support as used within the present invention may comprise a single or a mix of compounds within discrete predefined regions, or within an array of predefined regions wherein said predefined regions may or may not be physically separated from one another by a barrier that prevents lateral flow between predefined regions, wherein said compounds may be covalently or non-covalently attached within the porous structure of the porous solid support. The result of pulsing a liquid up and down through the support will be that a compound which is not attached to the support becomes mixed with said liquid. The volumes which are pumped through the porous support allow the spatial information to be maintained when barriers (microfluidic guides) between different spatial addresses are provided. When compounds are covalently or non-covalently attached within the porous structure of the solid support there is no need to provide spatial barriers since they retain their spatial coordinates during assay performance. For some applications it may be desirable to simultaneously test a plurality of mixed non-attached compounds (i.e. a pool of compounds) when pumping up and down through the porous support.

The present invention allows to test in parallel multiple test compounds in the presence of one another to assess desired or undesired combinations of drug-drug interactions.

The present invention provides for a high-throughput microarray analysis of direct or indirect interactions of molecular targets in a sample to an array of potential lead compounds.

The term “compound” or “potential lead compound” as used within the present specification refers to any molecule that can be screened in a drug discovery process.

Compounds

The methods and devices according to the present invention allow the use of a large diversity of libraries including biologically active libraries as well as libraries without disclosed biologically activity. Typical biologically active libraries are the so-called discovery libraries, targeted libraries and optimization libraries. The term “discovery library” refers to large size (>5000) members and has no preconceived notion about which molecular target(s) the compounds may be active against. The “targeted library” is biased in its design and refers to a library that contains a pharmacophore known to interact with a specific molecular target or family of molecular targets. The term “optimization library” refers to a library in which a lead exists and an attempt is being made to improve, for example, its potency, selectivity, or pharmacokinetic profile.

General suitable classes of compounds for use in the methods and devices according to the present invention include, by way of example and not limitation, natural compounds derived e.g. from plants with defined therapeutic applications, chemically synthesized compounds, compounds derived from combinatorial chemistry, peptide-based compounds, peptide derivatives and the like.

Biologically active libraries may include proteolytic enzymes such as for example serine proteases like trypsin, non-proteolitic enzymes including inducer molecules, chaperone proteins, antibodies and antibody fragments, agonists, antagonists, inhibitors, G-coupled protein receptors (GPCRs), non-GPCRs, and cytotoxic and anti-infective agents. Examples of libraries without disclosed biologically activity may include scaffold derivatizations, acyclic synthesis, monocyclic synthesis, bicyclic and spirocyclic synthesis, and poly and macrocyclic synthesis, or compounds which interact with any of the above-mentioned molecules.

In particular, test compounds, inducer molecules, chaperone proteins, hormones, oligopeptides, nucleic acids and synthetic variants thereof such as PNA's or LNA's, agonists, antagonists, inhibitors of cellular functions, enhancers of cellular functions, transcription factors, growth factors, differentiation-inducing agents, secondary metabolites, toxins, glycolipids, carbohydrates, antibiotics, mutagens, drugs, and any combination thereof are suitable compounds for use within the present invention.

Accordingly, in one embodiment of the present invention, compounds are chosen from the group comprising chemical compounds, natural compounds, oligo-peptide-based compounds, peptide derivatives, biologically active compounds, and any potential drug candidate compound.

Compounds obtained through combinatorial and so-called fast synthesis may be equally suitable.

In a further embodiment, said compounds are drugs selected from a chemical or natural drug candidate library.

In a particular embodiment of the present invention, methods are provided wherein compounds are chosen from the group comprising enzymes, enzyme substrates, inducer molecules, enhancer molecules, inhibitor molecules, chaperone proteins, transcription factors, differentiation-inducing agents, secondary metabolites, toxins, glycolipids, carbohydrates, antibiotics, mutagens, drugs, oligopeptides, nucleic acids, agonists, antagonists, aptamers, monoclonal and polyclonal antibodies, and any combination thereof. Said monoclonal antibodies may be engineered monoclonal antibodies or may be therapeutic monoclonal antibodies.

Delivery of Compounds to the Support

Delivery of compounds to predefined regions on the support may be accomplished by using a liquid handling device but may equally be accomplished by manual handling.

Accordingly, a liquid handling device may be positioned on the solid support, wherein said liquid handling device may be a high precision x-y-z pipettor or inkjet printer containing 1 or more channels through which liquid can be dispensed, sequentially or in parallel, to positions corresponding to predefined regions on the surface of the solid support.

Delivering of compounds may be by means of contact or non-contact spotting. The term “contact spotting” or “contact force” as used in this specification means a direct surface contact between a printing support and a delivery mechanism that may contain one or a plurality of guides such as in an array of tweezers, pins or capillaries that serve to transfer or deliver any content within the delivery mechanism to the surface by physically tapping said tweezer(s), pin(s) or capillary(ies) on the surface.

Compounds may also be delivered or spotted through ink-jet printing technology, a non-contact technology in which agents are sprayed onto the surface using technology adapted from computer ink-jet printers. The ink-jet method is sometimes called indirect because the agents are sprayed onto the surface rather than being directly placed. Ink-jet methods may be capable of producing smaller spots, and because they avoid physical contact with the surface may prove to be more reliable.

Useful ink-jet printing methodologies may include continuous and drop-on-demand ink-jet methods. Most suitable ink-jet printing methods are drop-on-demand ink-jet methods, examples of which include piezoelectric and electrostatic ink-jet systems.

Further useful in the present invention are spotting robots or liquid handling devices. Most spotting robots or liquid handling devices use an X-Y-Z robot arm (one that can move in three dimensions) mounted on an anti-vibration table. Said arm may hold nozzles in case of non-contact spotting. In contact spotting, said arm may hold pins. Nozzles or pins are dipped into a first micro plate to pick up the fluid to be delivered. The tips in case of pins are then moved to the solid support surface and allowed to touch the surface only minimally; the fluid is then transferred. The pins are then washed and moved to the next set of wells and fluid. This process is repeated until hundreds or thousands of compounds or molecules are deposited. Solid pins, quills, and pin-and-ring configurations of pins may be useful.

Alternatively, acoustic liquid transfer technology such as e.g. provided by EDC Biosystems (San Jose, Calif. 95131, United States) or Labcyte (former Picoliter Inc.; Sunnyvale, Calif. 94089, United States) may be used. These may be especially useful for transfer of liquid that may already contain precipitated matter.

Alternatively, a superposing mask comprising transversal holes may be superposed onto the support, wherein said superposing is such that each transversal hole in said mask corresponds to a predefined region on the surface of said solid support. As used in the present specification, a mask acts as a barrier to the lateral passage of a reagent. Typically, a pattern of holes in the mask allows selective passage of reagent and results in a corresponding pattern of reagent deposition on a surface placed behind/below the mask. A mask may be separate from the solid support and subsequently brought into contact with said solid support or may be part of the solid support (e.g. a barrier printed on the solid support). In the event that a mask is part of the support, said support may be compartmentalized by e.g. painting a masking agent onto said support. Criteria for suitable masking agents are (1) easy to apply, (2) low fluorescence, (3) resistant to solvent sterilization, (4) not water soluble and (5) capable of blocking both the pores in the support and preventing cross-flow between compartments. For example, latex liquid masking films with low fluorescence such as Talens Liquid Masking Film (052, Royal Talens, Apeldoorn, Holland) are suitable masking agents but other masking agents may be equally applicable.

Suitable superposing masks are made of inert material and prevent cross-transfer between predefined regions. Particular useful masks are penetrative and compartmentalize the porous solid support. For some applications there is no need for lateral passage between predefined regions to be prevented; i.e. predefined regions are not physically separated from one another in order for non-attached compounds to become mixed during assay performance.

Accordingly, in one embodiment of the present invention, deposition of the compounds is from above the support by a means chosen from the group comprising a delivery mask, a microfluidics device, a high precision x-y-z micro-pipettor, inkjet printer, acoustic liquid handling, and manual handling.

Delivery of compounds to the support may be by means of a contact force which may be a capillary force or a piezo-electric force.

Precipitation of some compounds during storage or handling is a recognized problem and known to occur with a large number of potent lead compounds. Due to the precipitation, often these compounds are excluded from screening programs because of the otherwise clogging of the liquid handling systems. A solution to this problem is provided by using a supply chamber or acoustic liquid transfer technology.

A supply chamber allows the delivery of reactants to the solid support which otherwise may suffer impracticalities; e.g. which may clog the capillaries of e.g. a spotting device, or needles or tips of a liquid handling device. A supply chamber as said gives access of its content to at least one array within an array of arrays to which it is attached by either physical attachment or by mechanical attachment or merely by being in liquid contact with the array. Physical attachment of the supply chamber to the solid support may be, by way of example and not limitation, heat- or laser-welding, latex masking agents, glues or chemical welding. Non-limiting examples of supply chambers in liquid contact with the support include gel patches and open capillaries.

A washing step usually follows to remove any possible toxic product that may be derived from the attachment procedure. Said physical and/or liquid contact is reversible and allows subsequent supply chambers with diverse contents to be combined with a same solid porous support. A removable supply chamber offers the advantage and flexibility of transferring compounds to the solid support and immediate interruption of said supply by removal of the chamber.

A suitable supply chamber comprises a planar square, rectangular or circular surface and an upstanding wall surrounding the circumference of said surface to form a chamber having an open top and a closed bottom surface. The open (top) end of the supply chamber is oriented towards the first or the second surface of the solid porous support to which it becomes then physically attached or attached by liquid contact with the array. Alternatively, a useful supply chamber may have one or more openings at top as well as bottom surface.

A useful supply chamber may comprise multiple-use-insertions for parallel studies. Multiple-use-insertions are optionally movable separations allowing the supply chamber to be compartmentalized. The spatial organization of the inserts determines the number of compartments and the number of arrays covered by one compartment. If no inserts are used, the supply chamber is likely to comprise one compartment.

Large compound libraries may be stored within a multiplicity of supply chamber compartments, ready for use in in vivo and in vitro screening assays

Compound libraries may be stored in the supply chamber after a drying treatment, after which they can be dissolved again, later on when an assay needs to be performed. Removal by drying of the solvent normally used to dispense the compounds provides the additional advantage of avoiding possible interference of said solvent with the subsequent assay. Upon compound dissolution; e.g. when in contact with an appropriate liquid or buffer, the compounds diffuse from the supply chamber into and through the pores of the porous solid support.

Depending on the solubility of the compound, diffusion may be total or partial and sufficient to allow for hit identification. Transfer of the compounds is not limited to diffusion, and may also be by pulsing a liquid sample back and forth through the porous support thereby maximising mixing of assay components. By pulsing a sample within the pores of the support, compounds in the supply chamber may be pulsed along.

Pulsing allows extremely good mixing even though the geometry might normally not allow proper mixing in miniaturized compartments. Pulsing may be particularly advantageously in bead-based assays that require proper mixing for the essential reactions (on the beads) to occur. An example is Yttrium silicate beads used in SPA. These beads precipitate without proper agitation which is very hard to accomplish in miniaturized containers.

Alternatively, compounds useful in the discovery process of drug candidates may be provided and stored within the porous structure of the solid support. Devices according to the present invention comprise a plate with an array of wells arranged in rows and columns, wherein the bottom of each well is a solid porous support with a plurality of through-going channels. Compounds may be dispensed into each of the wells and dried or concentrated into the porous support using e.g. slow evaporation, vacuum drying, freeze drying methods or by e.g. by blowing air or an inert gas such as e.g. helium above and below the wells.

Accordingly, it is another object of the present invention to provide a method for the manufacture of a compound-storage solid support comprising the steps of
(a) providing a solid porous support having first and second surfaces and at least one area with a plurality of through-going channels;
(b) providing compounds on said first or second surface of said solid porous support, said compound are in liquid condition and allowed to enter the porous structure within predefined regions of the said support;
(c) applying a drying treatment so as to bring said compounds in a dried or lyophilised condition, wherein said dried condition is obtained after a drying treatment by slow evaporation, vacuum drying or by blowing air or inert gas above and below said solid support; and
(d) storing said compound in dried condition within the porous structure of the solid support.

In one embodiment of the present invention, methods for the manufacture of a compound-storage solid support are provided wherein said drying treatment is by slow evaporation, vacuum drying or by blowing air or inert gas above and below said solid support.

In a further embodiment of the present invention, methods for the manufacture of a compound-storage solid support are provided wherein the compounds are chosen from the group comprising chemical compounds, natural compounds, oligo-peptide-based compounds, biologically active compounds, and any potential drug candidate compound.

In a further embodiment of the present invention, methods for the manufacture of a compound-storage solid support are provided wherein said compounds are drugs selected from a chemical or natural drug candidate library.

In a further embodiment of the present invention, methods for the manufacture of a compound-storage solid support are provided wherein said compounds are chosen from the group comprising enzymes, enzyme substrates, inducer molecules, enhancer molecules, inhibitor molecules, chaperone proteins, transcription factors, differentiation-inducing agents, secondary metabolites, toxins, glycolipids, carbohydrates, antibiotics, mutagens, drugs, oligopeptides, nucleic acids, agonists, antagonists, aptamers, monoclonal and polyclonal antibodies, and any combination thereof.

In a further embodiment of the present invention, methods for the manufacture of a compound-storage solid support are provided wherein said compounds are provided by deposition from above the support by a means chosen from the group comprising a delivery mask, a microfuidics device, a high precision x-y-z micro-pipettor, inkjet printer, acoustic liquid handling, and manual handling.

In yet a further embodiment of the present invention, methods for the manufacture of a compound-storage solid support are provided wherein said compounds are immobilized within the porous structure of the solid support by covalent attachment or by adsorptive attachment.

Alternatively, compounds may be immobilised directly or indirectly through a spacer into the pores of the solid support by using appropriate grafting chemistry; this may be irreversible or not.

These library plates may be stored until assay performance. Assays are directly performed in these compound plates by adding the appropriate buffers and further essential components, (e.g. enzymes and matching substrates) which then are flown through predefined regions on the solid porous support or pulsed up and down through the through-going channels.

Pulsing through the through-going channels of the porous solid support of a sample liquid provides for an improved mixing of compounds and sample molecular target(s), thus improving reaction kinetics.

The use of these compound plates avoids large-scale laborious and time consuming ‘just-in-time’ compound distribution processes and enhances critical time windows in which assays can be performed before enzymes or other assay components break down and become non-functional. A liquid sample is pumped up and down within the pores of the solid support and signals are measured by monitoring fluorescence, chemi-luminescence or by radiometric imaging.

Optionally, compound plates as disclosed herein may comprise a coating to affect slow or controlled drug release into the assay medium once the plate or the porous solid support is provided with buffer at the initiation of an assay. Such a coating finds particular use if a timely dosage of drugs into the assay medium is required over a longer period of time (e.g. with screening of whole organisms such as C. elegans or any other particular cellular screen in which drug delivery should be controlled or restricted in time). Another use of such a coating may be that it stabilizes the compounds. The coating may also combine both functions.

Accordingly, it is another object of the present invention to provide a solid porous support, characterized in that within its porous structure an array of compounds is provided, said compounds are stored in dried or lyophilised condition, wherein said dried condition is obtained after a drying treatment by slow evaporation, vacuum drying or by blowing air or inert gas above and below said solid support. Alternatively, said compounds within said porous structure may be stored in a gaseous or supercritical condition.

In one embodiment, a porous solid support according to the present invention is provided, wherein a supply chamber is further provided comprising an array of compounds within at least one compartment, said compounds are in a dried or liquid state. Alternatively, said compounds may be within at least one compartment of a supply chamber in gaseous or supercritical state.

Said compounds may be immobilized within said porous support e.g. by a spacer, i.e. covalent linkage. Alternatively, compounds may in some cases be directly covalently attached through derivatised moieties comprised within the compounds. Said spacer or derivatised moieties may contain a cleavable section that may be induced by a cleaving agent (chemically, by light, by enzymatic action, etc.) to establish the release of the bound compound.

The present invention also contemplates the use of a “master stock” from which more than 1 compound plate can be made. Typically, in corporate archival systems usually one or at least a very limited amount of stock tubes are maintained (usually in 96-tubes format). From these, occasionally a number of replicates of identical master stocks, i.e., aliquots of the mother stock, are prepared (in 96-, or 384-well format). These master stocks are maintained at less critical conditions and are used for a limited amount of time after which they are used up or discarded. From the master stocks, aliquots are taken and distributed into assay plates into which assays are performed (96-, 384-, 1536-well format). This is referred to in the body of the text as the compound distribution process.

Methods according to the present invention allow the preparation of several replicates of master stocks at a single point in time, thus obviating the need to make sequential withdrawal of aliquots from stock preparations with concurrent sequential opening of the original stock tubes and exposing them to moisture or potentially contaminating objects such as needles or pipet tips. Replicates made at one particular point in time are most likely to be chemically identical. This is in contrast with sequential replicate preparations over longer periods of time where one can no longer guarantee that the compounds are not (partly) precipitated or are not decomposed or have not taken up moisture. Replicates that are stored dry and in a conditioned environment are likely to be the same over time.

Molecular Target

In general, the term “molecular target” refers to any molecule or component that plays a key role in biological processes. Standard in vivo screening activities typically focus mainly on testing of compounds against whole organisms or cells. However, the availability of transfectable genome sequences, together with high-throughput screening technologies, is increasing the potential for discovering new drugs by conducting heterologous or transgenic screens based on molecular targets from organisms including for example intact cells; enzymes such as kinases, proteases, lipases, integrases and the like; enzyme substrates; receptors such as G-PCR receptors; ion-channels; lipids; metal induced/controlled proteins; aptamers; ribozymes; modified or non-modified nucleic acids; and monoclonal antibodies.

Targeting RNA or RNA analogues instead of proteins provides an alternative to targeting proteins. RNA as molecular target may provide a more specific binding and more sites may be accessible for interaction.

In one embodiment of the present invention, methods are provided, wherein a molecular target is chosen from the group comprising enzymes, enzyme substrates, oligo-peptides, proteins, RNA, receptors, ion-channels, lipids, carbohydrates, aptamers, ribozymes, nucleic acids, monoclonal and polyclonal antibodies, antibody fragments, and any derivatives and analogues thereof.

Labelling

Molecular targets useful in the present methods may be labelled to allow detection of compound-molecular target interactions.

Accordingly, in one embodiment of the present invention methods are provided, wherein a molecular target is a labelled molecular target.

Molecular targets may be pre-labelled by introduction of a luminescent indicator or may be labelled to allow radiometric assays.

Particular useful labels are fluorescent molecules including, by way of example and not limitation, fluorescein isothiocyanate (FITC), rhodamine, malachite green, Oregon green, Texas Red, Congo red, SybrGreen, phycoerythrin, allophycocyanin, 6-carboxyfluorescein (6-FAM), 2′,7′-dimethoxy-4′,5′-dichloro-6-carboxyfluorescein (JOE), 6-carboxy X-rhodamine (ROX), 6-carboxy-2′,4′,7′,4,7-hexachlorofluorescein (HEX), 5-carboxyfluorescein (5-FAM), N,N,N′,N′-tetramethyl-6-carboxyrhodamine (TAMRA), cyanine dyes (e.g. Cy5, Cy3), BODIPY dyes (e.g. BODIPY 630/650, Alexa542, etc), green fluorescent protein (GFP), blue fluorescent protein (BFP), yellow fluorescent protein (YFP), red fluorescent protein (RFP), and the like, (see, e.g., Molecular Probes, Eugene, Oreg., USA).

Compound-Molecular Target Interactions

The present invention provides methods of screening for agonist or antagonist activity of drugs, methods of characterizing their potency profiles, methods of identifying the receptor expression pattern of cell membrane exposed receptors (“receptor fingerprinting”), methods of determining toxicity profiles for the compounds (e.g. toxicological responses, CYP-450, HERC). The plurality of compound-molecular target interactions includes interactions selected from the group comprising of signal transduction, general protein-protein interactions, changes in enzyme activity, activation or inhibition of a receptor-mediated response, activation or inhibition of an ion channel, activation or inhibition of a second messenger pathway at a point downstream of a receptor or channel, toxicity, neuroprotection.

According to the methods of the present invention, the ability and potency of compounds to act as agonists or antagonists against cellular membrane-exposed receptors, ion channels, ion pumps, and ion transporters may be detected, evaluated and characterized.

Methods according to the present invention contemplate cellular screens as well as conventional in vitro screens.

Detection

Compound-target molecule interactions may be detected in a number of ways.

Compound-target molecule interactions may include a binding event which may be visualised by detection of the target molecule which may be a labelled protein. Said protein may be labelled either directly or indirectly, with e.g. an antibody, or a dye etc. Alternatively, on the level of the compound, a binding event may be visualised via generation of fluorescence proximal to the place where binding takes place (FRET, removal of quenching (such as with molecular beacons)). Further, detection may be by generation of a signal by enzymatic action or reduction in signal intensity by the presence of an unbound compound (competition principle).

Means for detecting signals in general are well known to those of skill in the art. Thus, for example, radiolabels may be detected using photographic film or scintillation counters, fluorescent markers may be detected using a photodetector to detect emitted illumination. Enzymatic labels are typically detected by providing the enzyme with an enzyme substrate and detecting the reaction product produced by the action of the enzyme on the substrate, and calorimetric labels are detected by simply visualizing the coloured label. Further detection means are for example (micro-)calorimetry and (light)-microscopy.

Particular useful detection of compound-molecular target interaction(s) within the methods of the present invention is by luminescence or fluorescence microscopy, regular light microscopy, electron microscopy, electro-chemiluminiscence (ECL), UV/VIS absorbance, microcalorimetry and radiometry. Said fluorescence microscopy may be time-resolved fluorescence and fluorescent correlation spectroscopy (FCS).

Accordingly, in one embodiment of the present invention, methods for compound screening are provided, wherein identification of compound-molecular target interaction is by a method chosen from the group comprising luminescence microscopy, regular light microscopy, electron microscopy, UV/VIS absorbance, microcalorimetry and radiometry.

In a further embodiment, methods for compound screening are provided wherein identification of compound-molecular target interaction is by luminescence, said luminescence is fluorescence, time-resolved fluorescence, lifetime fluorescence or electrochemiluminescence.

Similar to fluorescence, also phosphorescence provides a suitable detection means. Phosphorescence relates to a quasi-stable electron excitation state involving a change of spin state (intersystem crossing) which decays only slowly. It is similar to fluorescence, but the species is excited to a metastable state from which a transition to the initial state is forbidden.

Detection of compound-molecular target interactions may also be accomplished by multi-step detection practices. Said practices may be, by way of example and not limitation, sandwich assays as are well-known in the art and enzymatic conversions into a detectable product.

The present invention also contemplates the monitoring of more than one compound-molecular target interaction (i.e., multiplexing) by for example looking at fluorescence at different wavelengths by using e.g. CY3 and CY5 dyes, or by simultaneously employing different methods for detection.

In one embodiment of the present invention, assaying is performed in real-time.

In another embodiment of the present invention, assaying is conducted by end-point analysis.

Detector Molecules

Alternatively, detection may be by just visual inspection; or may be by the use of detector molecules. In the context of the present invention the term “detector molecule” refers to molecules which allow the detection of a compound-molecular target interaction. A detector molecule may also be generated by the conversion of a compound and/or molecular target.

Detector molecules may be already present within the porous structure of the solid support prior to initiating a screening procedure. Detector molecules may be provided within the porous structure of the porous solid support at predefined regions. Also, detector molecules may diffuse from a supply chamber into the pores of the porous solid support.

Accordingly, in one embodiment of the present invention, methods are provided wherein detector molecules are present within the pores of the solid porous support prior to initiating an assay.

Also, label may be quenched in the near proximity of the internal surface in the pores by e.g. a quencher doped into the pores. Subsequently a signal might become detectable only after conversion (different spectral properties) or distal monitoring (off the pores) of the assay product.

Examples of useful detector molecules include nucleic acids including modified analogues thereof, peptides and oligopeptides including modified analogues thereof, proteins, and antibodies including antibody fragments, enzyme substrates, carbohydrates and specific dyes.

Where detector molecules are not yet present within the porous solid support, compound-molecular target interactions may be assayed by the addition of the detector molecules to the support after incubation of compounds with the liquid sample comprising at least one molecular target.

Assaying of compound-molecular target interactions may be by:
(a) providing a detector molecule or detection agent to the porous solid support;
(b) optionally washing off excess of unincorporated detector molecule or detecting agent; and,
(c) detecting the presence or absence of a change in detectable signal, the presence of a change in detectable signal indicating a compound-molecular target interaction.

Alternatively, homogeneous mechanisms may be used as a means of signal detection. These do not require washing off the unincorporated detector molecules. In general, homogeneous assays, such as e.g. FRET-based assays, may be useful and do not require solid phase separations which are very common in e.g. filtration assays.

Solid Porous Support

As understood within present specification, the term “first and second surfaces of a support” refers to the outer top and bottom sides of said support. For a porous support, said first and second surfaces may therefore be physically distinct surfaces interconnected by an intermediate material having a plurality of through-going pores or channels or may be an integral part of a porous material.

A number of materials suitable for use as support in the present invention have been described in the art. Materials particularly suitable for use as support in the present invention include any type of porous support known in the art. More materials particularly suitable for use as support in the present invention include any type of solid porous supports known in the art. The term “porous support” as used in the present specification refers to a support possessing or full of pores, wherein the term “pore” refers to a minute opening or microchannel by which matter may be either absorbed or passed through. Particularly, where the pores allow passing-through of matter, the support is likely to be permeable.

It is understood that porous supports according to the present invention may be semi-porous. Semi-porous supports can be induced to become fully porous by e.g. a chemical treatment or an illumination treatment. The use of semi-porous supports is advantageous in particular if the mixing of (short living) components within a supply chamber compartment(s) and/or within the pores of the porous support in a synchronous manner at a certain time in an assay is envisaged or required.

The support may be in the form of porous beads, particles, sheets, films or membranes. For example, the support may consist of fibres (such as glass wool or other glass or plastic fibres), glass or plastic capillary tubes, or metal oxide membranes. The porous support may have simple or complex shape. The surface to which the molecule is adhered may be an external surface or an internal surface of the porous support. Particularly where the support material is porous, the molecule is likely to be attached to an internal surface. Where the solid support is porous, various pore sizes may be employed depending upon the nature of the system.

The material of the porous support may be, for example, a metal, a ceramic metal oxide or an organic polymer. As a metal, for example, a porous support of stainless steel (sintered metal) may be used. For applications not requiring heat resistance, a porous support of an organic polymer may also be used. Above all, in view of heat resistance and chemical resistance, a metal oxide may be used. In addition, metal oxides provide a support having both a high channel density and a high porosity, allowing high density arrays comprising different target molecules per unit of the surface for sample application. In addition, metal oxides are highly transparent for visible light. Metal oxides supports are relatively cheap that do not require the use of any typical microfabrication technology and, that offer an improved control over the liquid distribution over the surface of the support, such as electrochemically manufactured metal oxide membrane. Metal oxide membranes having through-going, oriented channels may be manufactured through electrochemical etching of a metal sheet.

According to one embodiment of the present invention, methods are provided wherein said solid support is a metal oxide solid support.

Further, in another embodiment of the present invention, methods for the manufacture of a compound-storage solid support are provided wherein said solid support is a metal oxide solid support.

The kind of metal oxide is not especially limited. Metal oxides considered are, among others, oxides of zirconium, mullite, cordierite, titanium, zeolite or zeolite analog, tantalum, and aluminium, as well as alloys of two or more metal oxides and doped metal oxides and alloys containing metal oxides.

Accordingly, in a further embodiment of the present invention, methods are provided wherein said metal oxide solid support is an aluminium oxide solid support.

Further, in another embodiment of the present invention, methods for the manufacture of a compound-storage solid support are provided wherein said solid support is an aluminium oxide solid support.

Metal oxide supports or membranes as employed in the methods of the present invention may be anodic oxide films. As well known in the art, aluminium metal may be anodized in an electrolyte to produce an anodic oxide film. The anodization process results in a system of larger pores extending from one face and interconnects with a system of smaller pores extending in from the other face. Pore size is determined by the minimum diameters of the smaller pores, while flow rates are determined largely by the length of the smaller pores, which can be made very short. Accordingly, such membranes may have oriented through-going partially branched channels with well-controlled diameter and useful chemical surface properties. Useful thicknesses of the metal oxide supports or membranes as employed in the methods and apparatuses of the present invention may for instance range from 50 μm to 150 μm (including thicknesses of 60, 70, 80, 90, 100, 110, 120, 130 and 140 μm). A particular suitable example of support thickness is 60 μm. A suitable support pore diameter ranges from 150 to 250 nm including 160, 170, 180, 190, 200, 210, 220, 230 and 240 nm. A particular suitable example of pore diameter is 200 nm. These dimensions are not to be construed as limiting the present invention.

Due to the characteristic porous structure of the solid supports according to the present invention minimal amounts of compounds may be deposited on its surface; e.g. an antifungal antibiotic that is active at concentrations below 1 microgram per millilitre can be printed on the surface of the porous solid support at 100 picograms per square millimetre and guarantee killing and germ tube inhibition of a fungal pathogen.

Advantageously, metal oxide membranes as described herein are transparent, especially if wet, which allows for assays using various optical techniques. WO 99/02266 which discloses the Anopore™ porous membrane or support is exemplary in this respect, and is specifically incorporated by reference in the present invention.

Particular useful porous supports as employed in the methods described in the present specification are 3-dimensional supports, which allow pressurized movement of fluid, e.g. the sample solution, through its structure. As such, particular useful porous supports as employed in the present methods possess a permeable or flow-through nature. In contrast with two-dimensional supports, 3-dimensional supports or microarrays as employed in the methods as described herein give significantly reduced hybridization times and increased signal and signal-to-noise ratios. Further, a positive or negative pressure may be applied to the arrays in order to pump the sample solution dynamically up and down through the support pores with a tight control of the volume pumped. Said dynamical pumping allows immediate removal and ability to perform real-time detection of generated products from a reaction which takes place within the pores of the support by fast binding of said generated products to the support pore walls. Flow-through supports are particularly suitable if the relation between the spatial position of a compound within the porous solid support and the signal is to be retained.

Accordingly, in one embodiment of the present invention, methods are provided wherein the solid support is a flow-through solid support.

Further, in another embodiment of the present invention, methods for the manufacture of a compound-storage solid support are provided wherein said solid support is a flow-through solid support.

The nature and geometry of the porous support as useful in the present invention will depend upon a variety of factors, including, among others, the type of array and the mode of attachment (e.g., covalent or non-covalent). Generally, the support according to the present invention may be composed of any porous material which will permit immobilization of a target-molecule and which will not melt or otherwise substantially degrade under the reaction conditions used.

Compound Attachment

The expression “immobilized” on a porous support” as used in the present specification refers to the attachment or adherence of one or more compounds to the surface of a porous support including attachment or adherence to the inner surface of said support.

In particular adhesion or adsorption of compounds to the inner surface of the support may be desired when the compounds are to be released into solution and become very well mixed.

Molecules or compounds may be immobilized either covalently (e.g., utilizing single reactive thiol groups or cysteine residues,) or non-covalently e.g., via immobilized antibodies, the biotin/streptavidin system, and the like, by any method known in the art. Where covalent immobilization is contemplated, the support should be polyfunctional or be capable of being polyfunctionalized or activated with reactive groups capable of forming a covalent bond with the target to be immobilized (e.g. carboxylic acids, aldehydes, amino groups, cyano groups, ethylenic groups, hydroxyl groups, mercapto groups and the like).

Further examples of the various methods that are available to attach compound molecules to porous supports include but are not limited to biotin-ligand non-covalently complexed with streptavidin, SH-ligand covalently linked via an alkylating reagent such as an iodoacetamide or maleimide which reacts with SH and forms a thioether bond, amine-ligand covalently linked via an activated carboxylate group (e.g., EDAC coupled, etc.), phenylboronic acid (PBA)-ligand complexed with salicylhydroxamic acid (SHA), and acrylic linkages allowing polymerization with free acrylic acid monomers to form polyacrylamide or reaction with SH or silane surfaces. More specifically, immobilization of proteins may be accomplished through attachment agents selected from the group comprising cyanogen bromide, succinimides, aldehydes, tosyl chloride, avidin-biotin, photo-crosslinkable agents including hetero bi-functional cross-linking agents such as N-γ-maleimidobutyryloxylsuccinimide ester (GMBS), epoxides, and maleimides. Antibodies may be attached to a porous support by chemically cross-linking a free amino group on the antibody to reactive side groups present within the support. For example, antibodies may be chemically cross-linked to a support that contains free amino, carboxyl, or sulfur groups using glutaraldehyde, carbo-di-imides, or hetero bi-functional agents such as GIVMS as cross-linkers.

So-called linker molecules may be useful in the application for spacing the compound molecules away from the activated support. Linkers may be long or short, flexible, semi-rigid or rigid, charged or uncharged, hydrophobic or hydrophilic, depending on the particular application. Particular useful linkers which may be present on the surface of a support and used for attachment of compound molecules to said surface are bifunctional, i.e. they will have one functional group or moiety capable of forming a linkage with the activated support and any other functional group or moiety capable of forming a linkage with another linker molecule or the compound molecule.

In certain circumstances, linkers may be used to “convert” one functional group into another. For example, an amino-activated support can be converted into a hydroxyl-activated support by reaction with, for example, 3-hydroxy-propionic acid. In this way, support materials which cannot be readily activated with a specified reactive functional group can be conveniently converted into an appropriately activated support. Chemistries and reagents suitable for “converting” such reactive groups are well known, and will be apparent to those skilled in the art.

Linkers may also be used, where necessary, to increase or “amplify” the number of reactive groups on the activated support. For this embodiment, the linker will have three or more functional groups. Following attachment to the activated support by way of one of the functional groups, the remaining two or more groups are available for attachment of target molecules such as oligonucleotides and polynucleotides. Amplifying the number of functional groups on the activated support in this manner is particularly convenient when the support cannot be readily activated with a sufficient number of reactive groups. Linkers may also be made to contain a cleavable bond that may be induced by a specific cleaving agent, such as e.g., a nucleic acid sequence that contains the recognition site for a restriction endonuclease, or a specific peptide (or protein) that contains the cleavage site for the corresponding peptidase (or protease).

Suitable linkers or cross-linker molecules include, by way of example and not limitation, polypeptides such as polyproline or polyalanine, saturated or unsaturated bi-functional hydrocarbons such as 1-amino-hexanoic acid, polymers such as polyethylene glycol, etc., 1,4-Dimethoxytrityl-polyethylene glycol phosphoramidites useful for forming phosphodiester linkages with hydroxyl groups and are described, for example in Zhang et al., 1991, Nucl. Acids Res. 19:3929-3933 and Durand et al., 1990, Nucl. Acids Res. 18:6353-6359. Other useful linkers are commercially available.

Although most applications may use in-solution compounds, it is contemplated that a compound may be covalently bound to the support. Covalent binding of an organic compound to a metal oxide is well known in the art, for example using the method described by Chu. C. W. et al. (J. Adhesion Sci. Technol., 7, pp. 417-433, 1993) and Fadda, M. B. et al. (Biotechnology and Applied Biochemistry, 16, pp. 221-227, 1992). Further, after activation of a metal oxide support by a silanating agent and binding of compounds, a number of amino-groups of said silanating agent can still be present as unloaded amino-groups. This may result in unwanted interactions of said amino-groups with various substances present in the medium in which the loaded support is used, resulting in high background signals. The unloaded amino-groups can be removed from the support without affecting the loaded part of the support by subsequently treating the loaded support with an acidic solution. Similarly, an activated and loaded support may be treated with a basic or neutral solution, provided that the method is not used for derivatization of aluminiumoxide nanoparticles aminated with (3-aminopropyl)-triethoxysilane, wherein the basic solution further contains a large excess of N-acetylhomocysteinelactone. In this regard, the application WO 01/12846 is exemplary, and is specifically incorporated in the present invention.

Accordingly, in one embodiment of the present invention, methods are provided, wherein a compound is immobilized by covalent attachment or by adsorptive attachment to said porous structure.

Quality Control

Methods and solid supports according to the present invention may have implemented a quality control marker to be used as a tool to confirm compound attachment, storage and reconstitution. Compounds may be looked at by optical inspection using standard absorbance or fluorescent-based detection technologies.

Extended quality control and maintenance (e.g., for precipitation or for water absorption into the DMSO solvent) which is normally applicable for large corporate chemical compound storage and archival systems is avoided within the methods of the present invention because the compounds are dry and not any longer in DMSO. This is a major money-and time consuming advantage improving directly the quality and integrity of the library.

Applications

The methods and devices according to the present invention are useful in a number of applications.

In one embodiment, the present invention provides for the use of methods as described herein for screening of compounds in the process of drug discovery.

It is a further object of the present invention to provide a kit for performing a method as provided by the present invention, comprising a solid porous support as provided by the present invention.

1. A method for screening of compounds for drug candidates comprising:
(a) providing a solid porous support having first and second surfaces and at least one area with a plurality of through-going channels; wherein said solid porous support comprises compounds within predefined regions of the said support; wherein said compounds within the porous structure are stored in dried condition, wherein said dried condition is obtained after a drying treatment by slow evaporation, vacuum drying or by blowing air or inert gas above and below said solid support;
(b) providing a liquid sample comprising at least one molecular target;
(c) mixing said dried compounds of step (a) with said liquid sample of step (b) by flow of the sample through said predefined regions of the solid support through the said through-going channels;
(d) screening said compounds for drug candidates; said screening is by monitoring in an assay a compound-target interaction by measurement of a signal, said signal indicating interaction between a compound and a molecular target;
(e) optionally screening for a compound having a putative effect on a drug candidate identified in step (d).

2. The method according to claim 1, wherein said compounds are chosen from the group comprising chemical compounds, natural compounds, oligo-peptide-based compounds, peptide derivatives, biologically active compounds, and any potential drug candidate compound.

3. The method according to claim 1, wherein said compounds are drugs selected from a chemical or natural drug candidate library.

4. The method according to claim 1, wherein compounds are chosen from the group comprising enzymes, enzyme substrates, inducer molecules, enhancer molecules, inhibitor molecules, chaperone proteins, transcription factors, differentiation-inducing agents, secondary metabolites, toxins, glycolipids, carbohydrates, antibiotics, mutagens, drugs, oligopeptides, nucleic acids, agonists, antagonists, aptamers, monoclonal and polyclonal antibodies, and any combination thereof.

5. The method according to claim 1, wherein deposition of the compounds is from above the support by a means chosen from the group comprising a delivery mask, a microfluidics device, a high precision x-y-z micro-pipettor, inkjet printer, acoustic liquid handling, and manual handling.

6. The method according to claim 1, wherein said compound is immobilized by covalent attachment or adsorptive attachment to said porous structure.

7. The method according to claim 1, wherein said molecular target is chosen from the group comprising enzymes, enzyme substrates, oligo-peptides, proteins, RNA, receptors, ion-channels, lipids, carbohydrates, aptamers, ribozymes, nucleic acids, monoclonal and polyclonal antibodies, antibody fragments, and any derivatives and analogues thereof.

8. The method according to claim 1, wherein said molecular target is a labelled molecular target.

9. The method according to claim 1, wherein said identifying of the compound-molecular target interaction is by a method chosen from the group comprising luminescence microscopy, regular light microscopy, electron microscopy, UV/VIS absorbance, microcalorimetry, and radiometry.

10. The method according to claim 1, wherein said luminescence is fluorescence, time-resolved fluorescence, lifetime fluorescence, or electrochemiluminescence.

11. The method according to claim 1, wherein said solid support is a flow-through solid support.

12. The method according to claim 1, wherein said solid support is a metal oxide solid support.

13. The method according to claim 12, wherein said metal oxide solid support is an aluminium oxide solid support.

14. The method according to claim 1, wherein said assaying is performed in real-time.

15. The method according to claim 1, wherein said assaying is conducted by end-point analysis.

16. The method according to any of claims 1 to 15, wherein detector molecules are present within the pores of the solid support prior to initiating an assay.

17. A solid porous support, characterized in that within its porous structure an array of compounds is provided, said compounds are stored in dried or lyophilised condition, wherein said dried condition is obtained after a drying treatment by slow evaporation, vacuum drying or by blowing air or inert gas above and below said solid support.

18. The solid porous support according to claim 17, wherein a supply chamber is further provided comprising an array of compounds within at least one compartment, said compounds are in a dried or liquid state.

19. A method for the manufacture of a compound-storage solid support comprising the steps of
(a) providing a solid porous support having first and second surfaces and at least one area with a plurality of through-going channels;
(b) providing compounds on said first or second surface of said solid porous support, said compound are in liquid condition; and allowed to enter the porous structure within predefined regions of the said support;
(c) applying a drying treatment so as to bring said compounds in a dried or lyophilised condition, wherein said dried condition is obtained after a drying treatment by slow evaporation, vacuum drying or by blowing air or inert gas above and below said solid support; and
(d) storing said compound in dried condition within the porous structure of the solid support.

20. The method according to claim 19, wherein said drying treatment is by slow evaporation, vacuum drying, or by blowing air or inert gas above and below said solid support.

21. The method according to claim 19, wherein said compounds are chosen from the group comprising chemical compounds, natural compounds, oligo-peptide-based compounds, biologically active compounds, and any potential drug candidate compound.

22. The method according to claim 19, wherein said compounds are drugs selected from a chemical or natural drug candidate library.

23. The method according to claim 19, wherein said compounds are chosen from the group comprising enzymes, enzyme substrates, inducer molecules, enhancer molecules, inhibitor molecules, chaperone proteins, transcription factors, differentiation-inducing agents, secondary metabolites, toxins, glycolipids, carbohydrates, antibiotics, mutagens, drugs, oligopeptides, nucleic acids, agonists, antagonists, aptamers, monoclonal and polyclonal antibodies, and any combination thereof.

24. The method according to claim 19, wherein said compounds are provided by deposition from above the support by a means chosen from the group comprising a delivery mask, a microfuidics device, a high precision x-y-z micro pipettor, inkjet printer, acoustic liquid handling, and manual handling.

25. The method according to claim 19, wherein said compounds are immobilized within the porous structure of the solid support by covalent attachment or by adsorptive attachment.

26. The method according to claim 19, wherein said solid support is a flow-through solid support.

27. The method according to claim 19, wherein said solid support is a metal oxide solid support.

28. The method according to claims 27, wherein said solid support is an aluminium oxide solid support.

29-31. (canceled)
